(19) United States
(12) Patent Application Publication
(10) Pub. No.: US 2008/0179544 A1
(43) Pub. Date: Jul. 31, 2008

(54) Particle therapy system

(76) Inventors: Werner Kaiser, Erlangen (DE); Tim Use, Nurnberg (DE)

Correspondence Address: BRINKS HOFER GILSON & LIONE, P.O. BOX 10395, CHICAGO, IL, 60610, US

(21) Appl. No.: 12/009,307

(22) Filed: Jan. 17, 2008

(30) Foreign Application Priority Data
Jan. 25, 2007 (DE) 10 2007 003 878.1

(51) Int. Cl. A61N 5/00

(52) U.S. Cl. 250/492.1

(57) ABSTRACT

A particle therapy system is provided. The particle therapy system may include a gantry that has a radiation unit and is rotatable about an axis of rotation. The gantry encloses a radiation chamber with a movable floor segment. A patient table is positionable in the radiation chamber. The movable floor segment is coupled to the gantry such that upon a rotation of the gantry, the floor segment remains in a horizontal zero position and as needed executes a motion about the axis of rotation of the gantry.

This patent document claims the benefit of German Patent Application No. DE 10 2007 003 878.1 filed on Jan. 25, 2007, which is hereby incorporated by reference.

### BACKGROUND

The present embodiments relate to a particle therapy system.

In particle therapy, especially in oncology, a particle beam, for example, having protons or heavy ions, is generated in a suitable accelerator. A beam channel guides the particle beam into a radiation chamber via an exit slot of the beam channel. The particle therapy system may include a beam exit slot that is stationary because of the complicated beam guidance. Alternatively, a particle therapy system may include a rotatable gantry with an exit slot. Because of the complicated beam guidance, the gantry as constructed is extremely bulky. The gantry encloses the approximately cylindrical radiation chamber, into which a patient table is moved. For precise treatment, the tissue of the patient to be irradiated is positioned in the isocenter of the system (for example, the point struck by the beam upon the rotation of the gantry).

A nozzle may be disposed directly in front of the exit slot, at the end of the beam channel. The nozzle may include at least one beam detector and passive beam element. The gantry is ideally rotatable about the patient by 360° to enable irradiating the patient from below. The radiation unit must be rotatable in the region below the patient. For that purpose, the floor of the radiation chamber opens and adapts to the rotation of the radiation unit.

International Patent Disclosure WO 2004/026401 A1 discloses a radiation chamber that is a half-open space the size of a room. The floor of this room is fixedly installed, except for a slit about 50 cm wide for guiding a radiation unit. The slit is covered with a rolling covering guided on both sides. The gantry is rotatable by only 180°.

European Patent Disclosure EP 1 402 923 A1 discloses a further particle irradiation device. The wall and floor of the radiation chamber include plates, which are joined and are pushed around the patient table upon rotation of the gantry. In this embodiment, the load-bearing capacity of the floor is greatly restricted.

### SUMMARY

The present embodiments may obviate one or more of the drawbacks or limitations inherent in the related art. For example, in one embodiment, a particle therapy system may irradiate a patient from all angular positions.

In one embodiment, a particle therapy system includes a gantry that has a radiation unit and is rotatable about an axis of rotation. The gantry encloses a radiation chamber with a movable floor segment. A patient table may be positioned in the radiation chamber. The movable floor segment is coupled to the gantry in such a way that upon a rotation of the gantry, the floor segment remains in a horizontal zero position and may rotate about the axis of rotation of the gantry, if needed.

The radiation unit may irradiate a patient from all angular positions because the floor segment and the gantry are supported relatively movably to one another. The floor segment is coupled to the gantry such that a rotation of the gantry does not necessarily move the floor segment. The term “floor segment” may include an element, which in the assembled state is in one piece and simply constructed and has a suitably selected mechanical load-bearing capacity. The floor segment may be solid or a hollow scaffold. The side of the floor segment that can be walked on is fixed and extends over the full surface (area) between the walls of the radiation chamber. If there is no risk of collision between the floor segment and the radiation unit, then the floor segment remains in the horizontal zero position, which assures access to the patient. In the event of a possible collision with the radiation unit, the floor segment is then displaced about the axis of rotation of the gantry, so that space is made available for the radiation unit beneath the patient table. For many irradiation angles (for example, up to +90°/−90°, beginning at a vertical location of the radiation unit above the floor segment), a stationary, gapless floor of sufficient load-bearing capacity in the radiation chamber is available, which assures access to the patient by workers and equipment. Only at irradiation angles of greater than +/−90° does the floor segment have to be moved out of the way.

In one embodiment, the particle therapy system may include a robot arm. The robot arm may place the patient table inside the radiation chamber. The robot arm may be secured to a solid floor located outside the radiation chamber. The robot arm may move the patient table into the radiation chamber enclosed by the gantry and hold the patient table in place without the patient table being in contact with the floor segment. To avoid a collision with the radiation unit, the floor segment may be moved out of the zero position without consideration of the position of the patient table.

The floor segment may be supported on the gantry. For example, the floor segment may be braced solely on the gantry. For movable support of the floor segment on the gantry, roller bearings are braced on a side wall of the gantry. Alternatively, the floor segment may be guided, for example, via a rail on the side wall of the gantry. The side wall may be a cylindrical jacket face including a load-bearing material and may define a boundary of the radiation chamber. Upon a rotation of the gantry, for moving the radiation unit that protrudes from the gantry about the axis of rotation, or upon a rotation of the side wall, the roller bearings roll along the moving side wall, so that the floor segment continues to remain in the horizontal zero position, without an external force being exerted. Displacement of the floor segment is under the influence of an external force. The floor segment is rolled to the side on the side wall by the roller bearings, or its position remains fixed relative to the side wall and it rotates with the gantry. The gantry is embodied such that the radiation unit, beginning at a vertical positioning above the floor segment located in the zero position, is rotatable by an angular range of +/−180° clockwise and counterclockwise about the axis of rotation, so that an angular range of rotation of 360° is covered.

The floor segment, viewed in cross section, may include a maximally semicircular geometry that is adapted to the gantry. The floor segment may be a solid circular-segmental body, or alternatively, it may be a circular-segmental frame that is hollow in cross section and that extends to below an isocenter of the gantry, to enable perfect positioning of the patient at the isocenter.

In one embodiment, a friction element firmly holds the floor segment in the horizontal zero position. Frictional engagement, or the firm hold of the floor segment with a frictional force, represents one easily attained possibility for holding the floor element in the horizontal zero position. The frictional force of the friction element may be greater than the frictional forces between the roller bearings and the side wall of the gantry, so that upon rotation of the gantry, rotation of the floor segment with the gantry is prevented without the exertion of an external force.

In one embodiment, the friction element is elastically supported. A small range of tolerances in the motion of the floor segment is created, because the frictional forces of the friction element act on the floor segment and return it to the zero position. The elastic support may be, for example, a spring or a solid body made of a flexible material, such as rubber.

In one embodiment, a cantilever connected to the floor segment is engaged by the friction element. The cantilever provides a large area for contact between the friction element and the floor segment. The large area for contact increases the range of tolerance or action of the friction element.

In one embodiment, a sliding-block guide extends all the way around the radiation chamber and guides the cantilever. The cantilever is disposed outside the radiation chamber, such that the cantilever has no direct contact with the moving parts of the gantry. The positioning of the patient table is not hindered by the cantilever and the cantilever can be firmly held, independently of the rotation of the gantry.

In one embodiment, the gantry is embodied such that the radiation unit, upon its rotation or at a defined rotary position of the floor segment, is pushed in the rotary direction. No additional drive or separate controller is necessary for moving the floor segment. The floor segment remains in the zero position until such time as it is carried along with the radiation unit in the rotation of the radiation unit and displaced in the rotary direction. The floor segment here is, for example, a lightweight metal construction, such that it does not represent a major additional load for the drive of the gantry, by comparison with the heavy radiation unit.

In one embodiment, a buffer element may be disposed on the radiation unit and/or on the floor segment, for example, in a region of a contact point between the radiation unit and the floor element. The buffer element may mitigate an impact between the radiation unit and the floor segment upon rotation of the radiation unit and assure a favorable gradual introduction of force into the floor segment.

The contact point between the radiation unit and the floor segment may be disposed outside the radiation chamber. The space in the radiation chamber is not unnecessarily reduced by the components such as buffer elements for embodying the contact point.

In one embodiment, two inclined vanes are provided on both sides of the floor element. The buffer element may be disposed on the vanes. The inclined position of each of the vanes makes a disposition of the buffer element possible in which a large-area contact takes place between the floor segment and the radiation unit. This increases the force into the floor segment.

In one embodiment, a back wall of the radiation chamber rotates with the gantry. This makes an economical, easily attained embodiment of the back wall possible, in the form of a lining that is not mechanically load-bearing.

In one embodiment, as an alternative to, or in combination with the rotating radiation unit pushing the floor segment forward in the rotary direction, a control unit may move the floor segment independently of the position of the radiation unit. A motion of the floor segment may be completely decoupled from the rotary motion of the radiation unit, so that a movement of the floor segment about the axis of rotation takes place even without direct contact between the floor segment and the radiation unit. This embodiment especially spares the gantry drive, which need not take on an additional load.

### BRIEF DESCRIPTION OF THE DRAWINGS

FIG. 1 shows a front view of one embodiment of a particle therapy system with a cylindrical radiation chamber; and

FIG. 2 shows a longitudinal section through the particle therapy system of FIG. 1.

### DETAILED DESCRIPTION

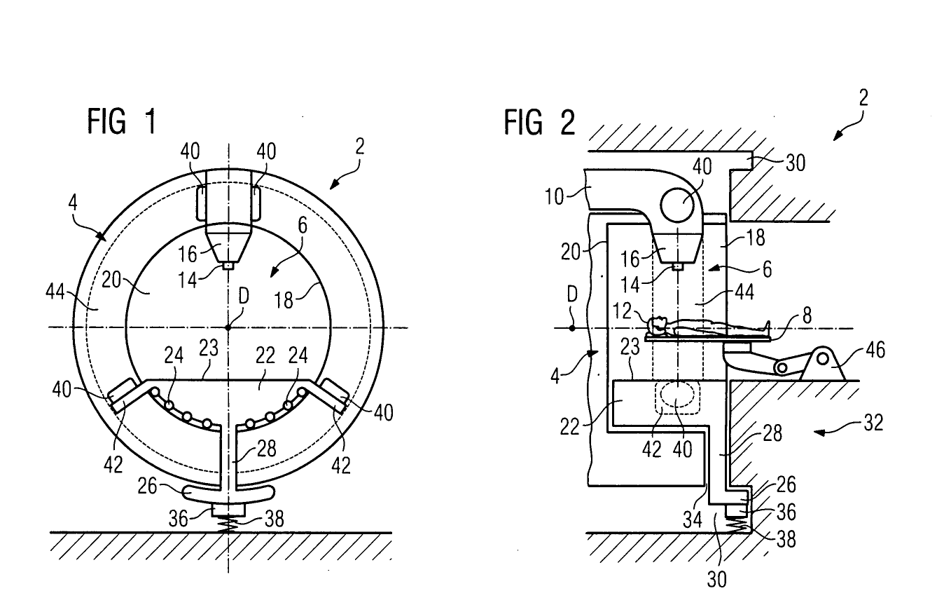

FIGS. 1 and 2 illustrate a particle therapy system 2 from different perspectives. The particle therapy system 2 may include a gantry 4 that is rotatable about an axis of rotation D. The axis of rotation D, in the view of the particle therapy system shown in FIG. 1, extends perpendicular to the plane of the drawing and is represented by a point D. The gantry 4 encloses an approximately cylindrical radiation chamber 6, in which a patient table 8 can be positioned, as can be seen from FIG. 2. The gantry 4 may include a beam channel 10. A particle beam, such as a heavy ion or proton beam, is guided in the beam channel 10 for treatment of a patient 12 lying on the patient table 8. The particle beam enters the radiation chamber 6 via an exit slot 14 of a radiation unit 16. The radiation unit 16 protrudes from a rotatable, load-bearing side wall 18 of the radiation chamber 6. To the rear, the radiation chamber 6 is bounded by a back wall 20, which may be a simple lining without mechanical load-bearing capacity. The back wall may rotate with the gantry 4 about the axis of rotation D.

The radiation chamber 6 may include a floor. The floor includes a single, solid floor segment 22 in the form of an arc, which is supported on the side wall 18 of the gantry 4. The floor segment 22 may assume a horizontal zero position and form a floor surface 23 that can be walked on, as shown in FIGS. 1 and 2. The load-bearing capacity of the floor segment 22 is selected to suit the requirements, so that the floor segment 22 forms a gapless radiation chamber floor that can be loaded with a weight of approximately 200 kg, for example.

The floor segment 22 may be supported on the side wall 18 via a number of bearings 24 such that the floor segment 22 and the side wall 18 are movable relative to one another. The bearings may be roller bearings. The gantry 4 may rotate while the floor segment 22 remains in its zero position. Alternatively, when the gantry 4 is stationary, the floor segment 22 may execute a motion about the axis of rotation D, driven by an external force by its bearings 24. In one embodiment, for moving the floor segment 22 about the axis of rotation D, for the floor segment's 22 position relative to the gantry 4 may remain fixed and for it to be slaved to the rotation of the gantry.

The floor segment 22 may include a cantilever 26. The cantilever 26 may connect to the floor segment 22 via a strut 28 that extends perpendicular to the horizontal floor surface 23. For guidance of the cantilever 26 and the strut 28 when the floor segment 22 moves about the axis of rotation D, the radiation therapy system 2 may include a sliding-block guide 30, extending all the way around the radiation chamber 6 and embodied in a solid floor 32, and a shaft 34, formed between the gantry 4 and the solid floor 32. For firmly holding the floor segment 22 in the zero position, the radiation therapy system 2 includes a friction element 36, which engages the cantilever 26 and is elastically supported via a spring 38.

As shown in FIGS. 1 and 2, the radiation therapy system 2 with a stationary floor may irradiate the radiation chamber 6 from different angles. For example, beginning at the vertical position shown of the radiation unit 16 above the floor segment 22, a rotation of the radiation unit 16 by 90° clockwise and counterclockwise is possible, without the threat of a collision with the floor segment 22. Within this angular range, the floor segment 22 does not be moved out of the way. Only at a deflection of the radiation unit 16 out of the position shown by an angle of rotation that is greater than +/−90° is a displacement of the floor segment 22 about the axis of rotation D effected. For that purpose, the floor segment 22 may have its own drive mechanism. Once triggered by a control unit, the drive mechanism may move the floor segment 22 about the axis of rotation D even before contact occurs between the floor segment 22 and the radiation unit 16. In the exemplary embodiment shown, a compulsory guidance of the floor segment 22 takes place, via the radiation unit 16. Upon rotation of the compulsory guidance, the floor segment 22 is pushed ahead of it.

In one embodiment, buffer elements 40 may be disposed on the radiation unit 16 and on the floor segment 22, in a region of a point of contact between the two. The buffer elements may prevent a hard impact between the radiation unit 16 and the floor segment. The floor segment 22 has two inclined vanes 42 on both sides, such that there will be a large area of contact between the floor segment 22 and the radiation unit 16. The vanes 42 extend outside the radiation chamber 6 and carry the buffer elements 40 of the floor segment 22. The inclined position of the vanes 42 provide an angle such that the buffer elements 40 of the floor segment 22 and of the radiation unit 16 rest on one another over a large area once the radiation unit 16, in its rotation, reaches the floor segment 22. The vanes 42 may be disposed outside the radiation chamber. Accordingly, an indentation 44 extends all the way around the side wall 18, as indicated in the drawings by a dashed line.

In one embodiment, the floor segment 22 includes a lightweight metal construction, so that the drive mechanism of the gantry 4 is loaded as little as possible upon displacement of the floor segment 22 by the radiation unit 16. Upon a deflection of the radiation unit 16 about an angle of deflection greater than 180°, of the force of gravity of both the floor segment 22 and the radiation unit 16, which act in the direction back to the zero position, are added together, which results in a high load on the drive mechanism of the gantry 4. The deflection of the radiation unit 16 may begin at the vertical position shown in the drawings. So that the drive mechanism will not be loaded unnecessary, the particle therapy system 2 may include a radiation unit 16, beginning at its vertical position, which may be moved only up to 180° clockwise or counterclockwise, but as a result the entire angular range of 360° is covered.

In one embodiment, the patient table 8 is moved into the radiation chamber 6 via a robot arm 46. The patient table 8 has no contact with the floor segment 22. The robot arm 46 is a multiaxial industrial robot arm with a multi-part mechanism and is mounted on the solid floor 32. The robot arm 46 may move the patient table 8 translationally in both the horizontal and the vertical direction. The robot 46 may rotate about a plurality of, such that the motion of the patient table 8 has three translational degrees of freedom and three degrees of rotational freedom. Using the translational and rotary motion of the patient table 8, the position and the removal of the patient 12 with respect to the radiation unit 16 are set. During positioning in the radiation chamber 6, the patient table 8 remains in a horizontal position, so that the patient 12 rests stably.

In one embodiment, the particle therapy system 2 may irradiate the patient 12 from all angular positions, and include problem-free movement of the radiation unit 16 below the patient table 8 because the floor segment 22, by way of its movable support, is pushed away from the radiation unit 16 itself. No additional drive mechanism and no separate triggering of the floor segment 22 are required. The floor segment 22 may remain in its zero position for most angles of irradiation, as a result of which there is a radiation chamber floor that may be walked on and driven on even during the treatment of the patient 12.

Various embodiments described herein can be used alone or in combination with one another. The forgoing detailed description has described only a few of the many possible implementations of the present invention. For this reason, this detailed description is intended by way of illustration, and not by way of limitation. It is only the following claims, including all equivalents that are intended to define the scope of this invention.

1. A particle therapy system, comprising:
a gantry that has a radiation unit and is rotatable about an axis of rotation, the gantry enclosing a radiation chamber
a movable floor segment; and
a patient table positionable in the radiation chamber,
wherein the floor segment is coupled to the gantry such that upon a rotation of the gantry, the floor segment may remain in a horizontal zero position and may rotate about the axis of rotation of the gantry.

2. The particle therapy system as defined by claim 1, wherein the floor segment is supported on the gantry.

3. The particle therapy system as defined by claim 1, wherein the floor segment, viewed in cross section, includes a maximally semicircular geometry that is based on the gantry.

4. The particle therapy system as defined by claim 1, comprising a friction element that holds the floor segment in the horizontal zero position.

5. The particle therapy system as defined by claim 4, wherein the friction element is elastically supported.

6. The particle therapy system as defined by claim 5, comprising a cantilever connected to the floor segment, the cantilever being engaged by the friction element.

7. The particle therapy system as defined by claim 6, comprising a sliding-block guide that guides the cantilever, the sliding-block guide extends around the radiation chamber.

8. The particle therapy system as defined by claim 1, wherein the gantry is embodied such that the radiation unit upon its rotation, beyond a defined rotary position, pushes the floor segment in the rotary direction.

9. The particle therapy system as defined by claim 8, comprising a buffer element disposed on the radiation unit and/or on the floor segment in a region of a contact point between the radiation unit and the floor segment.

10. The particle therapy system as defined by claim 9, wherein the contact point between the radiation unit and the floor segment is disposed outside the radiation chamber.

11. The particle therapy system as defined by claim 10, comprising two inclined vanes on both sides of the floor segment, the buffer element being disposed on the two inclined vanes.

12. The particle therapy system defined by claim 1, wherein a back wall of the radiation chamber may rotate with the gantry.

13. The particle therapy system as defined by claim 1, comprising a controller that is operable to move the floor segment independently of the position of the radiation unit.

14. The particle therapy system as defined by claim 1, wherein the movable floor segment is within the radiation chamber.
